(19) United States
(12) Patent Application Publication
(10) Pub. No.: US 2026/0256381 A1
(43) Pub. Date: Sep. 3, 2026

(54) MOVEMENT SCORE

(71) Applicant: Whoop, Inc., Boston, MA (US)

(72) Inventors: Emily Rachel Capodilupo, Boston, MA (US); Eric Robert Dougherty, Nashua, NH (US); Seul Ah Kim, Allston, MA (US); John Anthony Bowllan, Portland, ME (US); Victoria Harrison Lee, Waltham, MA (US)

(21) Appl. No.: 19/554,044

(22) Filed: Mar. 2, 2026

Related U.S. Application Data
(60) Provisional application No. 63/766,095, filed on Mar. 3, 2025.

(51) Int. Cl. A61B 5/11; A61B 5/00; A61B 5/0205; A61B 5/024

(52) U.S. Cl. CPC A61B 5/1118; A61B 5/0205; A61B 5/024; A61B 5/1126; A61B 5/6824; A61B 5/6826; A61B 5/7264; A61B 5/7271; A61B 5/742; A61B 5/7455; A61B 5/746; A61B 2560/0462; A61B 2562/0204; A61B 2562/0219; A61B 2562/0233

(57) ABSTRACT

Motion data is received from one or more motion sensors of a monitor worn by a user over a period of interest and is used to generate a movement score for the user based on a combination of active intervals and inactive intervals. This movement score can support real-time feedback and coaching, as well as long-term evaluation of a user's activity patterns.

### RELATED MATTERS

This application claims priority to U.S. Prov. App. No. 63/766,095 filed on Mar. 3, 2025, the entire content of which is hereby incorporated by reference.

This application is also related to U.S. application Ser. No. 19/461,616 filed on Jan. 27, 2026, which claims priority to U.S. Prov. App. No. 63/750,181 filed on Jan. 27, 2025, and International Patent App. No. PCT/US26/17269 filed on Mar. 2, 2026, which claims priority to U.S. Prov. App. No. 63/766,095 filed on Mar. 3, 2025, where the entire content of each of the foregoing is hereby incorporated by reference.

### TECHNICAL FIELD

This disclosure generally relates to techniques for scoring user movement using a combination of inactive and active periods within a time interval.

### BACKGROUND

Step count is a commonly used fitness metric for wearable fitness monitors. While counting steps is algorithmically straightforward and can provide a proxy for activity that has a known correlation to long-term wellness, step-based monitoring fails to account for other significant features of daily activity cycles, such as the duration, intensity, and timing of various activities during the day, as well as the negative effects of prolonged inactive periods. There remains a need for improved health metrics and measurements based on user movement.

### SUMMARY

Motion data is received from one or more motion sensors of a monitor worn by a user over a period of interest and used to generate a movement score for the user based on a combination of active intervals and inactive intervals. This movement score can support real-time feedback and coaching, as well as long-term evaluation of a user's activity patterns.

In one aspect, a computer program product comprises computer-executable code that, when executing on one or more computing devices, performs the steps of: continuously receiving motion data from one or more motion sensors of a fitness monitor worn by a user over a period of interest; performing a plurality of activity classifications with a machine learning model during the period of interest; identifying inactive intervals and active intervals within the period of interest based on the plurality of activity classifications; determining a maximum inactive period based on a greatest duration of contiguous inactive intervals during the period of interest; determining a total active time based on a sum of durations of the active intervals during the period of interest; calculating a movement score for the user based on a combination of the maximum inactive period for the user over the period of interest and the total active time for the user over the period of interest; assessing user activity for the period of interest based on the movement score; and generating an alert when the movement score meets a predetermined threshold.

The period of interest may be a current day. The alert may include a haptic notification to the fitness monitor. The steps may include adjusting the movement score based on one or more of the plurality of activity classifications and one or more corresponding activity intensities detected based on data from the fitness monitor.

In another aspect, a method described herein includes: receiving user data from one or more sensors of a monitor worn by a user over a period of interest; identifying inactive intervals within the period of interest; identifying active intervals within the period of interest; determining a maximum inactive period based on a greatest duration of contiguous inactive intervals during the period of interest; determining a total active time based on a sum of durations of the active intervals during the period of interest; calculating a movement score for the user based on a combination of the maximum inactive period for the user over the period of interest and the total active time for the user over the period of interest; and initiating a notification to the user based on the movement score.

The method may include displaying the movement score to the user. The method may include displaying movement metrics to the user including displaying one or more of the maximum inactive period, the total active time, and the movement score. The method may include providing coaching to the user on how to improve the movement score during a current day or a subsequent day. The method may include identifying the active intervals, which includes identifying motion patterns associated with known physical activity within the user data using a machine learning model. The machine learning model may include at least one of a gradient-boosted model for activity classification and a recurrent neural network with gated recurrent units for activity classification. The user data may include at least one of motion data and heart rate data. The method may include reducing a volatility of a calculation of the movement score at a beginning of a day for the user by adjusting a weighting of a contribution of the total active time to the movement score at the beginning of the day based on a number of measurement intervals for the day. Identifying the active intervals within the period of interest may include: performing an activity classification with a machine learning model a number of times during a measurement interval within the period of interest to provide a number of activity detections for the measurement interval; and identifying the measurement interval as an active interval when the number of activity detections for the measurement interval meets a predetermined threshold.

Calculating the movement score may include calculating a base score for the period of interest using a function based on a difference between the maximum inactive period and a target inactive period. Calculating the movement score may include calculating a base score for the period of interest using a first function that decreases as the maximum inactive period for the user increases. Calculating the movement score may include calculating a scaling factor for the period of interest using a second function that increases as the total active time increases. Calculating the movement score may include multiplying the base score by the scaling factor. Identifying the active intervals within the period of interest may include: performing an activity classification with a classification model a number of times over a measurement interval; dividing the measurement interval into a number of sub-intervals; identifying each of the number of sub-intervals as an active sub-interval when a number of activity detections in each of the number of sub-intervals meets a first predetermined threshold to provide a number of active sub-intervals; and identifying the measurement interval as an active interval when the number of active sub-intervals in the measurement interval meets a second predetermined threshold. The method may include progressively updating the movement score over a day for the user.

In another aspect, there is disclosed herein a system including: a physiological monitor including one or more sensors configured to continuously acquire user data for a user with the one or more sensors; and one or more processors configured by computer-executable code to receive data from the physiological monitor and to perform the steps of: receiving motion data from the one or more sensors over a period of interest, receiving heart rate data from the one or more sensors over the period of interest, identifying inactive intervals and active intervals within the period of interest based on a combination of the motion data and the heart rate data, determining a maximum inactive period based on a greatest duration of contiguous inactive intervals during the period of interest, determining a total active time based on a sum of durations of the active intervals during the period of interest, calculating a movement score for the user based on a combination of the maximum inactive period for the user over the period of interest and the total active time for the user over the period of interest, and initiating an alert to the user when the movement score meets a predetermined threshold.

The one or more processors may include a processor in the physiological monitor. The one or more processors may include a processor of a remote server coupled in a communicating relationship with the physiological monitor through a data network. The physiological monitor may include a wrist-worn monitor or a ring.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, with emphasis instead being placed upon illustrating the principles of the devices, systems, and methods described herein. In the drawings, like reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 is a flow chart illustrating a method for calculating a movement score.

FIG. 7 shows a user interface for presenting a movement score and associated metrics.

### DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein is not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods such as an hour, a day, or more (including acquisition throughout the day and night), etc. More generally with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics, such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user's body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example, increasing the sampling rate when signal variability increases, or during periods of relatively higher motion, or based on user input.

At the same time, continuous monitoring is not intended to exclude ordinary data acquisition interruptions, such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data, such as optical intensity signals, or processed data therefrom, such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to a non-transitory storage medium, such as storage hardware, storage devices, computer memory that may be accessed by a controller, a microcontroller, a microprocessor, a computational system, or the like, or any other module or component of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware, such as random access memory (such as DRAM, SRAM, EDO RAM), and so forth. Although not depicted, any of the devices or components described herein may include a computer-readable medium or other memory for storing program instructions, data, and the like.

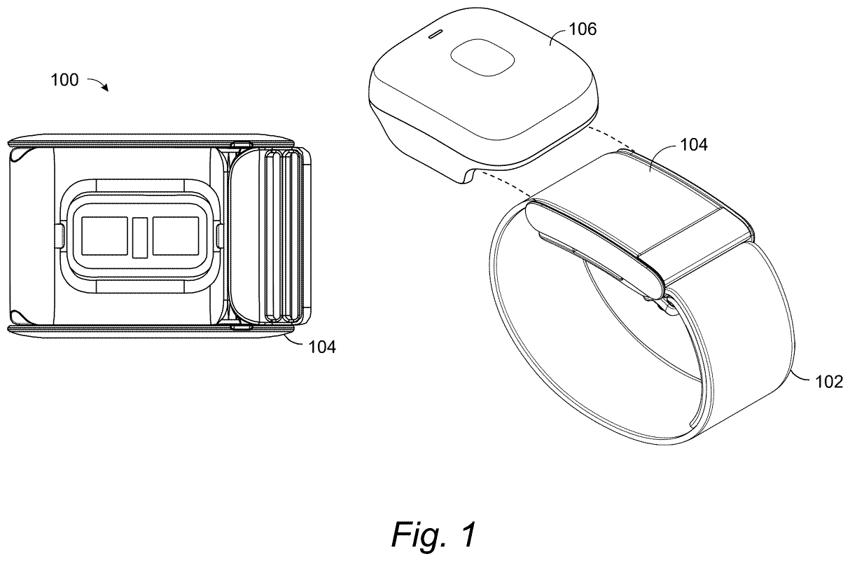

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as a rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 and U.S. Pat. Pub. No. 2024/0106283 describe non-limiting example embodiments of suitable wearable monitors 104, and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104 such as data collection, processing, display, and communications with external resources. For example, the system 100 may include hardware for a heart rate monitor using, e.g., photoplethysmography, electrocardiogra any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by or through the wearer's skin, or depending on the configuration, through capillaries or arteries.

The system 100 may be configured to record other physiological and/or biomechanical parameters, including, but not limited to, skin temperature (using a thermometer), galvanic skin response (using a galvanic skin response sensor), motion (using one or more multi-axes accelerometers and/or gyroscopes), blood pressure (via physical pressure measurements or other means), sound, electrocardiograms, and the like, as well as environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting additional functions of the wearable monitor 104. For example, the system 100 may include communications systems to support, e.g., near-field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a GeoPositioning System (GPS), a display and/or user interface, a clock and/or timer, and so forth.

The wearable monitor 104 may include one or more sources of battery power, such as a first battery within the wearable monitor 104 and a second battery 106 that is removable from and replaceable with the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium-range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

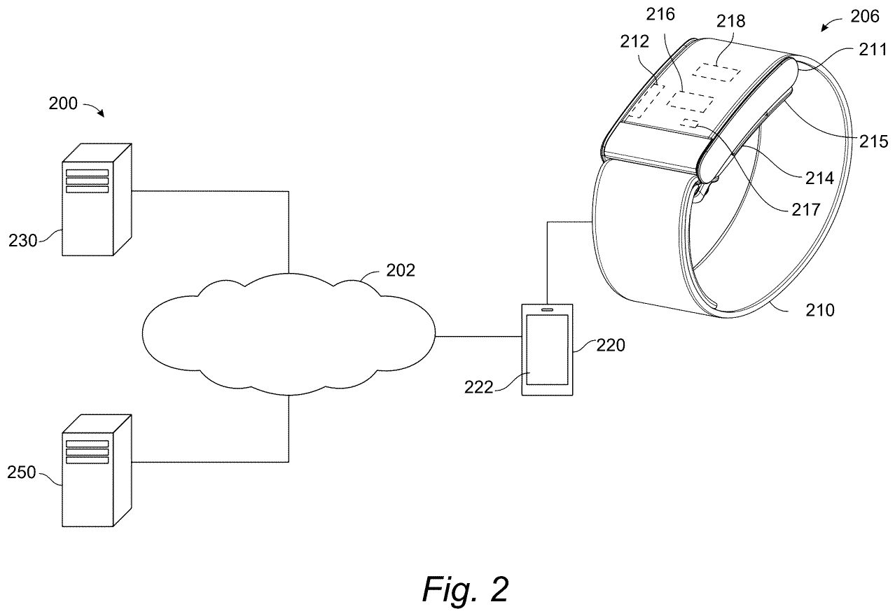

FIG. 2 illustrates a physiological monitoring system. More specifically, FIG. 2 illustrates a system 200 for physiological monitoring that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communication with local resources. By way of non-limiting examples, short-range communications may include Wi-Fi communications, Bluetooth communications, infrared communications, near field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

The network interface 212 of the physiological monitor 206 may be configured to couple the physiological monitor 206 to one or more other components of the system 200 in a communicating relationship, either directly, e.g., through a cellular data connection or the like, or indirectly through a short-range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength-specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211, or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and/or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery, sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, the physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors, such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200, such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may, for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicate detected motion, and/or beacons or the like to self-identify type, weight, and so forth, in order to facilitate automated detection and tracking of exercise activity with other connected devices.

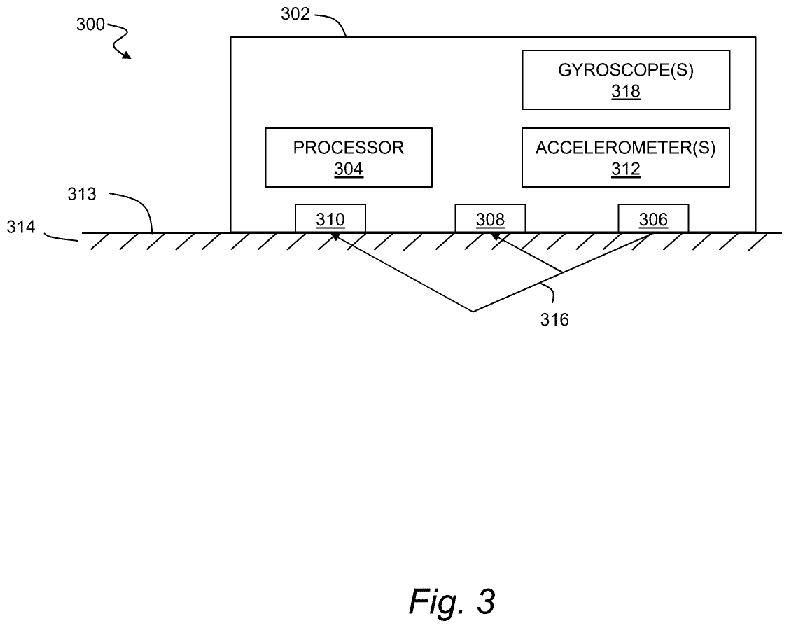

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like, such as audio earbuds, earrings or similar, glasses and/or other eyewear, a ring, a headband, and so forth.

The processor 304 may be any microprocessor, microcontroller, application-specific integrated circuit, or other processing circuitry or combination of the foregoing suitable for controlling operation of the physiological monitor and acquiring physiological data.

The light source 306 may include one or more light-emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316 (or transmitted through the tissue to one or more opposing sensors), where the intensity can be measured. In one aspect, the light source 306 may include light-emitting diodes that emit light in the green, red, infrared, near-infrared, or other suitable wavelength ranges, which can provide desired light transmission through human skin, facilitating low-power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1-4 millimeters from the light source 306 and the second sensor 310 may be positioned 2-8 millimeters from the light source, or about twice as far as the first sensor 308 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors 308, 310 may be linearly arranged in a straight line away from the light source 306. While this provides consistency in comparative measurements, it is not strictly required, and the sensors 308, 310 may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, the two sensors 308, 310 may be positioned at different distances from a perimeter of the physiological monitor 302 so that the sensors 308, 310 can acquire differential intensity values for ambient light incident on the skin and transmitted through the skin to the sensors 308, 310.

In operation, the processor 304 may acquire raw intensity data from the sensors 308, 310, and perform local calculations such as pre-processing raw data for heart rate measurements, or evaluating whether the physiological monitor 302 is properly placed for use on the skin 314.

The accelerometer 312 may include, e.g., one or more single-axis or multi-axis accelerometers, which may usefully measure motion of the physiological monitor 302 to support calculations such as automated activity detection, device on/off evaluation, degree of musculoskeletal activation, and so forth. Other motion and orientation sensing hardware-such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

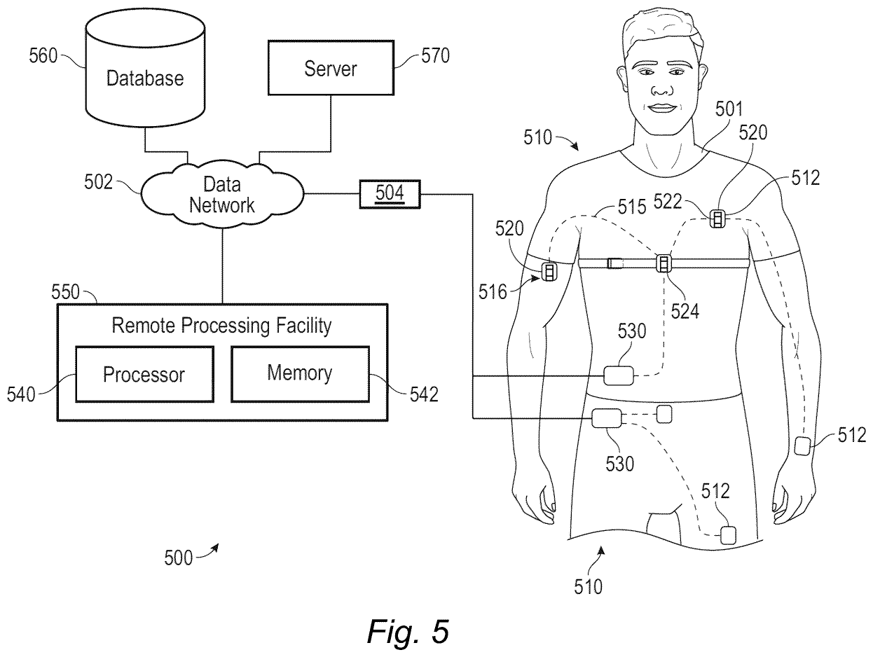

The physiological monitors described herein—e.g., in the systems 100, 200, 300 described above or elsewhere herein—may be provided in one or more different form factors. That is, although a wrist-worn device is illustrated in FIGS. 1 and 2, and garments with sensors are illustrated in FIG. 5, other form factors are also or instead possible, some of which are discussed below by way of example.

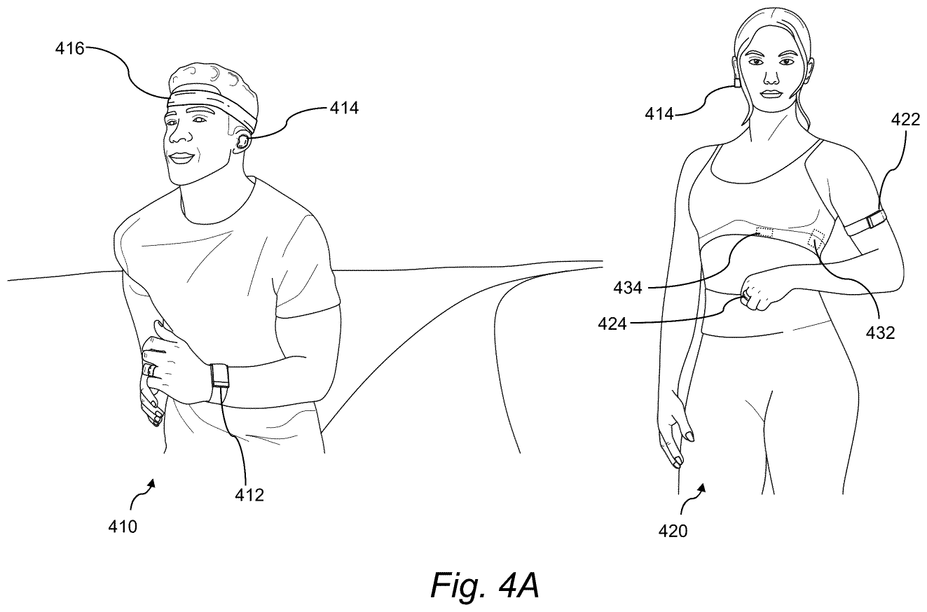

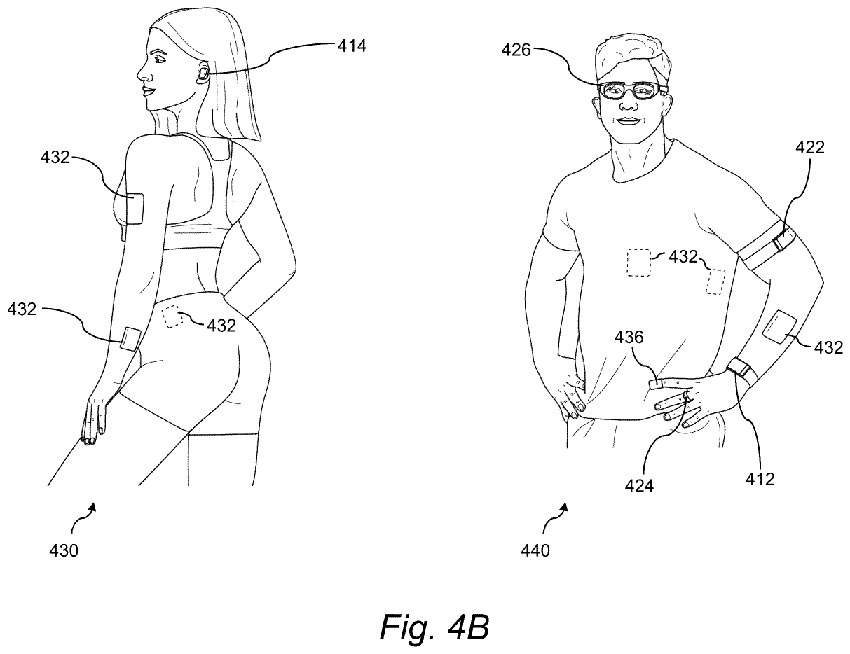

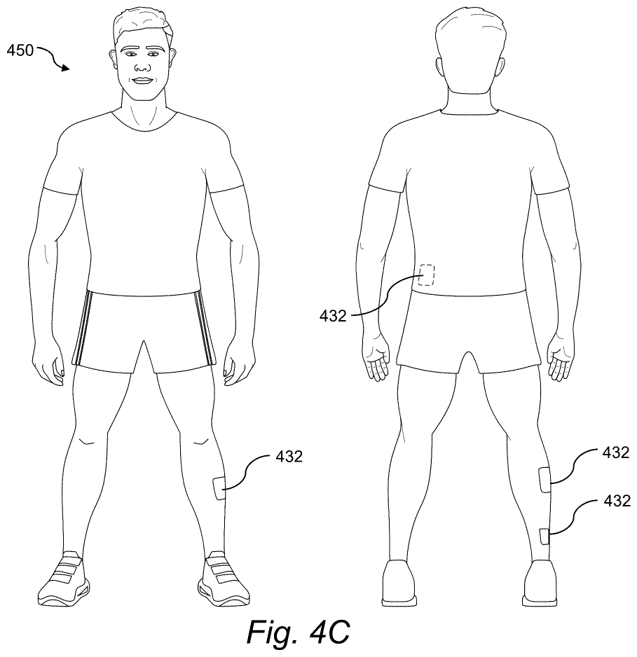

FIGS. 4A-4C illustrate physiological monitoring devices. The illustrated devices may include any of the hardware, software, and/or other components described herein for physiological sensing and/or other functions, and may be embodied in various form factors for various use cases. These various form factors may be used individually or as multiple independent or cooperating physiological monitoring devices, and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1-3 above.

FIG. 4A shows a first user 410 and a second user 420. The first user 410 may be wearing one or more physiological monitors, such as a wrist-worn device 412 (such as any described herein), an ear-worn device 414 (including on-ear devices retained with a clamp, clip, or other mechanism, and/or in-ear devices, such as earbuds or the like, that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring, such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health, and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large-scale motion, such as geographical movement and elevation changes, that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors and may be more suited to, e.g., wrist bands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to as pulse oximetry or SpO2) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein, may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands, neck bands, wrist bands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions, and may be configured for gesture control of physiological monitoring and/or related hardware and software. The ring 424 may be configured for wearing on a finger, as shown in the figure, or another portion of a wearer's body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smartphone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a finger-tip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The finger-tip monitor 436 may include a clamp, clip, or the like, and may be the same or similar to any of the physiological monitors described herein. In some aspects, the finger-tip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smartphone or other computing device, to perform cooperative monitoring. For example, various monitoring techniques, such as electrocardiography blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as Wi-Fi or cellular signals, and converting them into usable electrical power), ambient light harvesting, and so forth. Other techniques may also or instead be used to provide external power, such as beam steering or resonant techniques for short-range or medium-range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware, and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system may be embedded in a sleeve cuff for interactions with a payment terminal. Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on-capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smartphone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interaction, for example by storing data locally and transmitting the data over available local area network resources when a local access point such as a wireless access point or a relay device (such as a laptop, tablet, or smartphone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within the one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiography, electroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user's skin, and one or more light detectors for receiving light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements, such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities, while heart rate data may preferentially be gathered near major arteries. In another aspect, some measurements, such as temperature, may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly, where packet loss, latency, and the like may present challenges to real-time processing.

The communications interface 524 may be any as described herein, for example, including any of the features of the network interface 504 described above.

The controller 530 may be configured, e.g., by computer-executable code or the like, to determine a location of the module 520. This may be based on contextual measurements such as accelerometer data from the module 520, which may be analyzed by a machine learning model or the like to infer a body position. In another aspect, this may be based on other signals from the module 520. For example, signals from sensors such as photodiodes, temperature sensors, resistors, capacitors, and the like may be used alone or in combination to infer a body position. In another aspect, the location may be determined based on a proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500, such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510, and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed, such as machine learning models, lookup tables, analytical models, or the like, which may be applied to accelerometer data to detect an activity type. Other motion data, such as gyroscope data, may also or instead be used, and activity recognition processes may also be augmented by other potentially relevant data, such as data from a barometer, magnetometer, GPS system, and so forth. This may generally discriminate, e.g., between being asleep, at rest, or in motion, or this may discriminate more finely among different types of athletic activity, such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510, and then use this detected location to select a suitable model for activity recognition. This technique may similarly be applied to calibration models, physiological signals processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes and may, for example, be used to improve the design or use of modules 520 and garments 510, either for a population, for a user type, or for a particular user.

As stated above, the system 500 may further include a processor 540 and a memory 542. In general, the memory 542 may bear computer-executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like, as shown in the figure. Thus, in an aspect, one or more of the processor 540 and the memory 542 are included on at least one of the plurality of modules 520. In this manner, processing may be performed on a central module or on each module 520 independently. In another aspect, one or more of the processor 540 and the memory 542 are remote relative to each of the plurality of modules 520. For example, processing may be performed on a connected peripheral device, such as a smartphone, laptop, local computer, or cloud resource.

The processor 540 may be configured to assess the quality of the data received from a physiological sensor 522 of the module 520, or otherwise processed data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500 such as any discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources may advantageously be used in combination to improve physiological monitoring and achieve modes of monitoring not previously available.

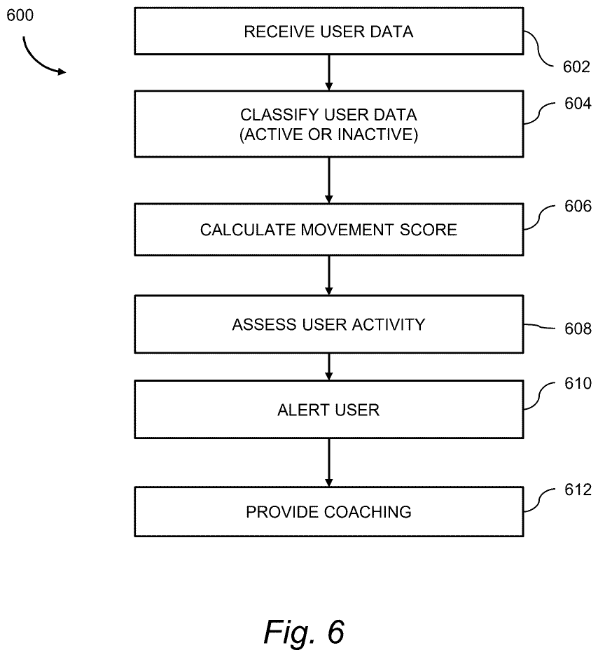

FIG. 6 is a flow chart illustrating a method 600 for calculating a movement score. The method 600 may be used in cooperation with any of the devices, systems, and methods described herein, such as by a user device (e.g., a mobile device) that is communicatively coupled to a wearable, continuous physiological monitoring device. For example, the method 600 may be used with the one or more user devices 220 that are communicatively coupled to the physiological monitor 206, and/or with one or more remote servers 230 that provide remote data processing resources (such as any of the processors or processing resources described herein), as illustrated in FIG. 2. More generally, the method 600 may be performed locally (e.g., using a processor disposed on a wearable physiological monitor), using an intermediate device such as a user device (e.g., using a processor disposed on a smartphone or the like), remotely (e.g., using a processor of a remote server or other remote processing resource), or any combination thereof. In general, the method 600 uses a movement score based on, e.g., metrics objectively associated with all-cause mortality, and supports coaching to the user based on the movement score and other data associated with the user. The movement score may change over time as the distribution of inactive time and active time changes over the course of a period of interest, such as over the course of a current day. The movement score may also be adjusted based on data associated with the user.

In one aspect, the movement score can be used to gamify movement, e.g., by encouraging users to regularly break up inactive periods while minimizing demotivation experienced by a user when they are not reaching a goal. The movement score may generally be positively affected by movement of the user, and coaching or other recommendations may be provided, e.g., when desired minimum movement is not being achieved. For example, the movement score or associated metrics may be associated with one or more achievements, levels, milestones, challenges, or rewards. A user may be presented with visual, auditory, or haptic feedback when certain thresholds are reached, such as by interrupting an inactive period prior to exceeding a target maximum inactive period or indicating the achievement of a predetermined amount of total active time. In one aspect, the movement score may be used to generate competitive or cooperative challenges, including comparisons to prior performance of the user, comparisons to a selected demographic group, or participation in group-based goals. The movement score may also be associated with point accumulation, streak tracking across multiple periods of interest, or unlocking of progressive targets based on historical performance. In this manner, the movement score may be used not only as a quantitative assessment of inactive and active behavior, but also as a mechanism for incentivizing behavioral change and sustained engagement with the movement scoring framework. This is further enabled by the real-time calculation and assessment of the movement score, allowing users to change and modify their behavior in real time, rather than just planning to change behaviors and habits at some time in the future.

As shown in step 602, the method 600 may include receiving user data over a period of interest. Receiving user data may include continuously receiving data such as motion data from one or more motion sensors of a fitness monitor worn by a user over a period of interest. Thus, for example, motion data may include data from one or more accelerometers, one or more gyroscopes, or one or more other motion sensors or the like. More generally, the user data may be obtained from sensors of a wearable physiological monitor or any of the other monitors described herein that might be used to distinguish between active and inactive states. Thus, user data may also or instead include physiological data such as heart rate data, skin temperature data, respiration rate data, and so forth, which may be acquired from, e.g., optical sensors, electrical sensors, audio sensors, temperature sensors, and so forth. In another aspect, user data may be acquired from external sensors such as cameras, motion detectors, proximity sensors, and the like, which may be used to detect and/or characterize movement by a subject over a period of interest. More generally, while movement data is useful for classification of user activities and for distinguishing between active and inactive intervals, it will be understood that other sensors may also or instead be used to classify intervals as described herein. In another aspect, the user data may include data received from sources other than the wearable fitness monitor, such as a user input or medical records. The user data may include user data over a period of interest or data outside the period of interest, e.g., where historical user data is used to evaluate current movement.

The period of interest for calculating a movement score may be a prior day, e.g., where historical movement scores are desired or stored for a user. The period of interest may also be a current day, e.g., to provide a current movement status for the user. The period of interest may be a current awake interval for the user, e.g., where a real-time or nearly real-time movement score is to be provided to the user for immediate feedback and coaching. The period of interest may also or instead span a plurality of hours, days, weeks, months, or years. In one aspect, the period of interest may be an aggregation of consecutive or non-consecutive periods of time. For example, a movement score may usefully be calculated for a preceding four weekend days or a preceding ten business days in order to distinguish between weekday and weekend activity cadence.

As shown in step 604, the method 600 may include classifying user data using any suitable movement classification models or the like. For example, this may include performing a plurality of activity classifications with a machine learning model during a period of interest. For example, inactive intervals and active intervals within the period of interest may be identified by receiving sensor data such as accelerometer data and/or other physiological data from a wearable device and classifying a user state into activities using any suitable combination of data, algorithms, machine learning models, and so forth. In general, inactive periods are sedentary or physically idle periods with little or no motion or exertion, while active periods are periods of physical activity.

In this disclosure, an “active” interval is generally a time interval during which sensor data from a monitor worn by a user indicates physical activity above a predetermined activity threshold, such as detectable body motion and/or a physiologic response consistent with exertion, as determined from one or more motion sensors and optionally one or more physiological sensors. An “inactive” interval is generally a time interval during which the sensor data indicates a sedentary state or other idle or low exertion state below the predetermined activity threshold, including substantially reduced body motion and optionally a physiologic response consistent with rest such as a heart rate or respiration approaching resting values. In some embodiments, the predetermined activity threshold is implemented by a classification model that outputs an activity label, an activity intensity, or an active-versus-inactive classification for each measurement interval, and the measurement interval is identified as active when a number of active detections within the measurement interval meets a predetermined threshold and otherwise is identified as inactive. More generally, any suitable classification criteria, rules, tools, or the like may be used consistent with sorting user states into generally active and generally inactive states during a period of interest.

In some embodiments, activity classification may be performed using one or more machine learning techniques that transform motion data and/or heart rate data acquired by a physiological monitor into one or more activity labels, activity intensities, or binary classifications such as inactive versus active. The motion data may include, by way of example, tri-axial accelerometer signals and/or tri-axial gyroscope signals. The heart rate data may include, by way of example, beat-to-beat intervals, heart rate time series, heart rate variability (HRV) features, and/or raw or filtered photoplethysmography (PPG) waveforms. The machine learning techniques described below may be used individually or in combination (e.g., in an ensemble), and may execute locally on the monitor, on a companion user device, on a remote server, or in a distributed manner.

In one aspect, activity classification using data from a physiological monitor may be performed with feature-based supervised learning models operating on windowed sensor streams. In such approaches, motion data (e.g., tri-axial accelerometer and/or gyroscope signals) and optionally heart-related data (e.g., heart rate, beat-to-beat intervals, or heart rate variability features) are segmented into fixed-duration measurement windows, features are computed, and a classifier such as logistic regression, a support vector machine, a random forest, or a gradient-boosted decision tree model outputs an activity label, an activity intensity, or an inactive-versus-active classification. These models can be computationally efficient and well suited for on-device deployment.

In another aspect, sequence-aware models may be used to impose temporal consistency and capture time-dependent patterns in activities. For example, hidden Markov models or related probabilistic sequence models may use an emission model that produces per-window activity likelihoods and a transition model that discourages implausible rapid state changes. Neural sequence models, such as recurrent neural networks (including GRU or LSTM architectures), may process sequences of motion samples, optionally fused with aligned heart-related signals, to learn temporal dynamics, such as gait periodicity, transitions between activities, and physiologic lag between motion onset and heart rate rise.

Deep learning models may also or instead be implemented using convolutional or attention-based architectures that learn representations directly from multi-axis time-series signals. One-dimensional convolutional neural networks and temporal convolutional networks may learn local and multi-scale temporal patterns from raw or lightly processed motion and/or heart signals. Transformer-based time-series models may use attention mechanisms to capture longer-range context that can help disambiguate activities with similar short-term signatures, and may be deployed as lightweight, distilled, or quantized models depending on compute constraints.

In another aspect, multi-modal fusion techniques may combine motion and heart-related data to improve robustness, such as reducing false positives due to incidental motion without physiologic response or heart rate elevation without corresponding physical activity. Classification may be further improved through personalization and domain adaptation, such as adapting a population-trained model to a particular user or device form factor, and through self-supervised or semi-supervised learning to leverage large unlabeled datasets. Post-processing techniques, such as majority voting, hysteresis, minimum-duration constraints, and smoothing of probability outputs, may be used to stabilize classifications for downstream use, such as inactive bout detection and movement scoring.

More generally, a variety of machine learning techniques may be used to generate a stream of initial classifications of activity based on data from a monitor. In one aspect, a collection of these activity classifications may be analyzed over a window to improve accuracy and consistency of resulting classifications. Thus, for example, identifying inactive intervals and active intervals within the period of interest may include performing an activity classification with a classification model a number of times over a measurement interval. Identifying inactive intervals and active intervals within the period of interest may include performing a non-linear transformation of average motion data from one or more motion sensors to detect physical activity. In this manner, activities may be classified according to, e.g., various types of sports activities and the like, such as stretching, calisthenics, running, walking, tennis, swimming, rock climbing, bicycling, and so forth. These classifications may be used, in turn, either alone or in combination with other user data and metrics, to determine when a user is active and when a user is inactive over any particular window, so that a chronological record of inactive versus active time for the user can be compiled. These classifications may also be used to determine or infer an intensity of any movement during measurement intervals for the period of interest.

It will be understood that, while workouts provide one useful group of activity types suitable for classification, in the context of this disclosure, activity classification may also or instead include a variety of daily activities. For example, activities other than workouts or exercise that can be classified based on motion detection with a wearable monitor include driving or riding in a vehicle, typing or computer work, reading, watching television, gaming, cooking or food preparation, washing dishes, doing laundry, vacuuming, sweeping, mopping, making a bed, showering or bathing, brushing teeth, shaving, handwashing, eating, drinking, standing in line, commuting on public transit, shopping or grocery browsing, carrying items, climbing stairs as part of routine movement, sitting in meetings, desk work, phone calls, childcare tasks (e.g., rocking or holding a child), pet care (e.g., feeding or walking a pet in a non-workout context), household tidying, and meditation or other seated breathing sessions. Tracking these types of activities can help to distinguish between inactive and active intervals even where a subject is not engaged in a formal exercise (such as tennis, swimming, jogging, etc.).

As a significant advantage, using specific activity classification as a factor when determining when a user is active and is inactive can improve detection of inactive and active periods by reducing misclassification in cases where, e.g., instantaneous motion data fails to distinguish between low-motion sport activities and true inactivity. More generally, using a machine learning model to identify inactive and active intervals provides an improvement in accuracy of classifying activity intervals compared to other methods, such as classifying intervals using steps of a user based on the motion data, by capturing complex motion patterns and time-based patterns that are not reflected in step counts alone, thereby reducing misclassification, including but not limited to low-movement activities, stationary exercises (e.g., bicep curls and planks), and prolonged inactive states with incidental motion.

For intervals where no activity is identified and/or no motion is detected, the intervals may be classified as inactive intervals. In general, the intervals may be intervals of any suitable length. For example, in one embodiment, each measurement interval may be a four to nine minute interval (e.g., a five minute interval) or similar, and the classification of inactive versus active may be a function of what percentage of the interval is affirmatively identified as active. Thus, for example, a five minute time interval may be considered active if at least a certain amount of the interval, e.g., two to three minutes of the example five minute interval, is identified as active. It will be appreciated that other intervals may also or instead be used depending on the desired accuracy and the available compute and memory resources. It will also be appreciated that the threshold for activity may be higher or lower. Thus, for example, an interval may be considered active when at least 50% of the interval is identified as active, at least 60% of the interval is active, at least 75% of the interval is active, and so forth.

Other methods may also or instead be used to classify a measurement interval. For example, the measurement interval may be divided into a number of sub-intervals and each sub-interval may be identified as an inactive sub-interval or an active sub-interval using the methods previously described for classifying a measurement interval. The measurement interval may then be classified based on whether a number of inactive sub-intervals over the measurement interval meets a predetermined threshold, or based on whether a number of active sub-intervals over the measurement interval meet a second predetermined threshold. The predetermined threshold(s) may be determined and/or adjusted based on at least one of: physiological data, movement data, a movement score, a recovery score, a sleep score, an input received by the user, data from a third party, and an assessment of the user.

For example, the measurement interval may be an interval of four to nine minutes (e.g., five minutes), with five minutes providing a useful sampling window for characterizing activity versus inactive states in a manner that usefully averages user states to smooth measurements and simplify downstream calculations, while also helping to ensure that brief but meaningful periods of activity-a quick walk or stretch—are properly accounted for as breaks in extended inactive periods. An interval may be identified as active if the number of activity detections in the measurement interval meets a predetermined threshold. In another scenario, an interval such as a five-minute interval may be divided into sub-intervals, such as five one-minute sub-intervals, or another range such as four overlapping two-minute intervals, as two to four minutes. Each sub-interval may be identified as active or inactive using any suitable classification or strain calculation, and the threshold for activity may be that two out of five contiguous one-minute sub-intervals or two out of four overlapping two-minute sub-intervals are identified as active intervals. Thus, in one aspect, a five-minute interval may be identified as active if two or more contiguous one-minute sub-intervals in the five-minute interval are identified as active. The predetermined threshold may be varied based on context. For example, the predetermined threshold may be increased when a user is consistently meeting fitness goals that are tracked using the physiological monitor, or decreased when a user is not consistently meeting fitness goals.

In one aspect, a user input may be used, either alone or in combination with user data such as motion data, to identify a measurement interval or a set of measurement intervals as active. For example, a user input may indicate that the user is starting an activity, e.g., yoga, weightlifting, or running. This user input may be used to assist in classification of measurement intervals, thereby significantly reducing the processing power required for classification. In another aspect, the user input may be used in combination with other data and calculations to aid in the classification of measurement intervals, thereby providing benefits such as increasing processing efficiency, decreasing processing time or complexity, and improving classification accuracy. For example, the user may indicate they are starting a yoga workout. This user input may be used in combination with motion data to classify measurement intervals with a machine learning model for measurement intervals. In general, each measurement interval may be tagged with a specific activity, tagged categorically as inactive or active, or both.

In some embodiments, an inactive interval may be modified for movement score calculations by excluding or reclassifying the interval based on context so that the scoring better reflects meaningful activity or inactivity. For example, an interval classified as inactive may be excluded when the system detects a nap or sleep-related recovery state, such that a 45-minute nap that helps to recover from a sleep debt does not increase the maximum inactive period for the day. As another example, a user may enter an indication of an activity (e.g., stretching from 2:00-2:20 PM), and the system may retroactively reclassify those intervals as active so that the activity breaks an inactive bout that would otherwise reduce the movement score.

In some embodiments, an inactive interval may be weighted or otherwise adjusted, rather than fully excluded, to account for intervals that are inactive but less relevant to negative outcomes or are uncertain due to data quality. For example, a meditation session detected or entered by the user may remain labeled as inactive but be down-weighted (e.g., counted as 15 minutes of inactive time for scoring even if 30 minutes elapse). As another example, an off-wrist gap may be excluded from inactive time calculations, or inactive intervals immediately before and after the off-wrist gap may be merged into a single continuous inactive period (e.g., merging 12:00-12:55 PM and 1:10-1:45 PM across a 12:55-1:10 PM off-wrist interval) to prevent artificial “breaks” in inactive bouts.

In some embodiments, an inactive interval may be refined using higher-resolution information to more accurately detect genuine interruptions. For example, where measurement intervals are 5 minutes, a brief but qualifying interruption (e.g., 90 seconds of walking within an otherwise inactive 5-minute slice) may be used to split or truncate the inactive interval so the interruption breaks the inactive bout for purposes of maximum inactive period. As another example, physiological corroboration may be used to avoid false breaks, such that incidental wrist motion without a corresponding physiologic response (e.g., heart rate remaining consistent with rest) does not reclassify an inactive interval as active for movement score calculations.

In one aspect, if the wearable physiological monitor, or other monitor, was not worn for a period of time by the user in a period of interest, the intervals before and after the period of time may be excluded, weighted, and/or otherwise modified based on the identification of intervals before and after the period of time when the wearable physiological monitor is not worn. For example, the inactive intervals before and after the period of time when a wearable physiological monitor was not worn may be combined into a single inactive period. The active intervals before and after the period of time when a wearable physiological monitor was not worn may be combined into a single active period.

In one aspect, user data may be used to select the method for classifying a measurement interval. For example, where user data indicates that the user is engaging in activities associated with time-based data with low displacement, a recurrent neural network configured to model sequential motion patterns may be selected. Where user data indicates that classification is to be performed on summarized features within fixed measurement intervals and/or on a device with limited computational resources, a gradient boosted model or other feature-based classifier may be selected. In another example, where the user input indicates a start time and an end time for an activity, the user input may be relied on to identify an activity without the processing of any data received from a monitor.

In another aspect, user data may be used to select or adjust one or more parameters associated with the classification method. For example, a predetermined threshold for identifying an active sub-interval within a measurement interval may be adjusted based on a user's historical activity level, baseline heart rate, or prior inactive patterns. A number of activity detections required to classify a measurement interval as active may be increased for users consistently exceeding movement score targets or decreased for users with limited mobility. Similarly, weights applied to motion magnitude, heart rate data, or other sensor inputs may be adapted based on user-specific physiological responses to activity. In this manner, the classification of each measurement interval may be personalized to improve distinction between inactive and active states for the individual user, or to improve the quality of active/inactive decisions in marginal cases for a five-minute interval.

In another aspect, user data may be used to dynamically switch between classification methods during a period of interest. For example, during intervals associated with sleep or recovery, a first classification model configured for low-motion sensitivity may be employed, while during typical waking or workout intervals, a second classification model configured for identifying the type of an active activity may be used. User data such as device type, sensor placement, battery state, or network availability may also influence whether classification is performed locally using a computationally efficient model or remotely using a higher-complexity model. By selecting the classification method and/or parameters based on user data, the system may improve classification accuracy, reduce misclassification of inactive and active intervals, and enhance the reliability of downstream movement score calculations.

As shown in step 606, the method 600 may include calculating a movement score as described herein. The movement score may be calculated based on a combination of the maximum inactive period for the user over the period of interest and the total active time for the user over the period of interest.

In embodiments, a movement score is calculated from sensor-derived activity interval classifications over a period of interest. Motion data may be continuously received from one or more motion sensors of a monitor worn by a user as described herein, and a plurality of activity classifications may be performed with a machine learning model or using other techniques during the period of interest. Based on the activity classifications, the system identifies inactive intervals and active intervals within the period of interest, determines a maximum inactive period as a greatest duration of contiguous inactive intervals during the period of interest, and determines a total active time as a sum of durations of the active intervals during the period of interest. The movement score for the user can then be calculated based on a combination of the maximum inactive period and the total active time. Qualitative assessments and coaching may then be derived based on the quantitative movement score, including generating or initiating a notification or alert when the movement score meets a predetermined threshold.

In certain claimed embodiments, calculating the movement score includes calculating a base score for the period of interest using a first function that decreases as the maximum inactive period for the user increases. A base score may be calculated based on the maximum inactive period using a formula that quantitatively emphasizes the positive effects of avoiding prolonged inactive states. Studies have shown that prolonged periods of sedentariness have been identified as a risk factor for chronic illness. Health guidelines recommend that inactive intervals should consistently and frequently be interrupted by periods of movement. The base score may thus reflect these risks of prolonged inactive activity while motivating a user to become more active and reduce sedentariness, and in particular, continuous sedentariness. In one aspect, the base score may be calculated based on a maximum inactive period during the period of interest, along with a target maximum inactive period based on a desired limit for periods of inactivity. For example, in one embodiment, the base score may be expressed as a function based on a difference between the maximum inactive period (Sₘₐₓ) and the target maximum inactive period (S_{target}). The function may thus quantify inactive behavior during a given time frame in an understandable manner for a user—e.g., where the base score is a non-linear representation that provides a nuanced representation of the relationship between inactive time and health impacts. The function may include one or more of a sigmoid function, an exponential function, a piecewise linear function, a logarithmic function, a polynomial function, a step function, and the like. By way of example, the S_{target} may change based on the user's base score's proximity to 1. For example, if the user is able to continually achieve the S_{target}, the S_{target} may be changed to become increasingly difficult to achieve. Inversely, if the user is not able to continually achieve the S_{target}, the S_{target} may be changed to become easier and easier to achieve. For example, the S_{target} may be set as 85% of the average Sₘₐₓ for the past week. Periods of time other than a week may be used to calculate the average Sₘₐₓ. The S_{target} may be changed based on an input received from the user and/or any data associated with the user. The base score may be continually recalculated over a period of interest as more data for the temporal distribution of active time and inactive time is revealed. The base score may also be recalculated for a period of interest based on user inputs that provide additional context for the base score.

By way of non-limiting example, the base score may be calculated using an asymmetrical custom sigmoid function such as:

$$base ( x ) = \{ \begin{matrix}1 - tanh ( ( x - {S}_{target} ) * 2 ) & if x < target \\ 1 - tanh ( \frac{( x - {S}_{target} )}{2} ) & otherwise\end{matrix}$$

where S_{target} is the desired limit for the duration of inactive periods and x is the maximum observed inactive period. This approach yields a base score of 1 when the maximum observed inactive period is equal to the desired limit, S_{target}. At the same time, this approach to calculating the base score usefully escalates the base score quickly when the maximum observed inactive period is below the desired limit, while penalizing a user more gradually as the maximum observed inactive period exceeds the desired limit.

In another aspect, a scaling factor may be applied to the base score based on periods of activity by a user. In this manner, a user can recover from a poor base score through prolonged periods of exercise or other physical activity. In general, active intervals may be determined based on the classification of data over the period of interest as described herein. A total active time for a user during a period of interest may be determined. In an embodiment, the total active time for a user during a period of interest may be based on the sum of the durations of the active intervals during the period of interest. A weighting of an active interval used in a calculation may be adjusted based on the activity associated with the active interval, or upon other factors such as heart rate, amount of motion, and so forth. Thus, for example, a weighting of an active interval may be adjusted based on a magnitude of movement associated with the active interval, a strain calculated for the active interval, an average heart rate for the interval, and so forth.

In general, the scaling factor may quantify a mitigating factor for inactive periods by measuring the proportion of waking hours spent in an active state that is detected, e.g., using the techniques described herein. For example, the scaling factor can enable a user to achieve a movement score greater than the base score that is derived from the maximum inactive period (or some other base score metric), e.g., to recover in part from a poor score that results from an Sₘₐₓ less than their S_{target}. The scaling factor may be based on an interval of time, the number of intervals of time over a period of time, the total number of intervals of time over a period of interest, or any other measures of activity that can be used to mitigate inactive periods. An activity score, strain score, or other assessments and data may also or instead be used to evaluate activity and support a scaling factor as contemplated herein. In one aspect, the scaling factor may be based on the total active time in a period of time of interest. In another aspect, the scaling factor may be based on the target inactive period (e.g., a desired limit on inactivity). In an embodiment, the scaling factor may be expressed as a function of an interval of time, the number of intervals of time over a period of time, the total number of intervals of time over a period of interest, and the target maximum inactive time.

By way of non-limiting example, the scaling factor may be calculated based on each time slice in an active state, active (t), using a formula such as:

$$scale = \frac{{\sum }_{t}^{N} active ( t )}{\frac{N}{12}} - \frac{1}{{S}_{target}}$$

This scaling factor can help to counteract some of the base score penalty for inactive intervals by giving credit for each additional time slice spent in an active state (excluding time slices required to keep inactive intervals within the desired limit). In another aspect, the scaling factor may be based on a sum of active intervals, a ratio of active to inactive intervals, or some other formula that captures the mitigating impact of intermittent activity. This technique may be further modified to adjust the scaling based on the duration, intensity, and type of activities performed during the day. Thus, for example, for a user who runs a marathon, it may not make sense to penalize that user for prolonged rest for the remainder of the day. Similarly, the scaling formula may permit tiers of activity so that more intense activities increase the scaling factor (per unit of active time) more than less intense activities. More generally, the scaling factor may be adjusted, weighted, or otherwise calculated to account for the beneficial effects of different types, intensities, and durations of exercise.

The total movement score may be calculated as a product of the base score, f(x), and the scaling factor, scale. For example, calculating the movement score may include calculating a base score for the period of interest using a function based on a difference between the maximum inactive period and a target inactive period as described herein. More generally, calculating the movement score may include calculating a base score for the period of interest using a first function that decreases as the maximum inactive period for the user increases, and calculating a scaling factor for the base score using a second function that increases as the total active time increases. Other data may also be used to calculate the movement score. Calculating the movement score may include multiplying the base score by the scaling factor, or otherwise adjusting or weighting the base score according to the scaling factor.

In one aspect, e.g., when a user first awakes and the maximum inactive interval is zero, a calculated movement score may change frequently and significantly during the beginning of the period of interest. During this interval, small changes in the data can yield large changes in calculated values. To address this volatility, the method may include de-weighting the scaling factor in the beginning of the period of interest. The weighting of the contribution of the scaling factor may increase over time as accumulated data increases. By the end of this transition or initiation interval for the period of interest, the scaling factor (and the base score) may be fully weighted such that corrections or adjustments are no longer required to capture the active-inactive scoring strategy described above. Thus, in one aspect, calculating a movement score may include reducing a contribution of the scaling factor during an initial measurement period such as the beginning of the period of interest. Calculating the movement score may also or instead include reducing a volatility of a calculation of the movement score at the beginning of the day for the user by adjusting a weighting of a contribution of the total active time to the movement score at the beginning of the day based on a number of measurement intervals for the day. Calculating the movement score may also or instead more generally include reducing the weight of a contribution of the total active time to the movement score during the beginning of a period of interest. In another aspect, the scaling factor may be modulated during the first 4 hours after waking to reduce volatility as a user establishes a first meaningful inactive period.

In another aspect, calculation of the movement score may be adjusted based on data associated with the user. Adjusting the movement score may include adjusting the movement score based on one or more of a plurality of activity classifications and one or more corresponding activity intensities detected based on data from the fitness monitor. The calculation of the movement score may be adjusted to personalize operation of the scoring model. User data may include demographic information, historical movement patterns, baseline physiological metrics, recovery scores, sleep metrics, typical activity preferences, fitness level, mobility limitations, and prior movement score trends. One or more parameters used in calculating the movement score, such as a target inactive period, a weighting applied to active intervals, a functional form of a base score, a scaling factor coefficient, or a threshold for classifying intervals as active, may be modified based on such user data. For example, a target inactive period may be increased or decreased relative to a population baseline based on a user's historical maximum inactive durations, and weighting of active intervals may be adjusted according to user-specific heart rate response or strain metrics. In one embodiment, the relative contribution of the base score and scaling factor may be adapted over time based on user performance trends to promote engagement by the user and achievable progression. By adjusting scoring parameters based on user data, the movement score may more accurately reflect individual physiology and behavior, reduce variability between users, and provide feedback that is tailored to the user.

In general, the movement score may be progressively updated over the day as additional intervals are classified and accumulated. Thus, a real-time, or substantially real-time, movement score may be presented to the user that provides current information about whether a user's patterns of inactive and active states for a current day are promoting good health and longevity. This can advantageously relieve a user of tracking incremental intervals of inactive and active states, and provide a movement score, based on real-time tracking data for the user, that penalizes prolonged inactive intervals while rewarding mitigating intervals of activity. Thus, the movement score as described herein can promote healthy daily routines based on, e.g., all-cause mortality data, by providing instant and up-to-date information summarizing the health consequences of a current day's activities in a user-interpretable, quantitative score.

As shown in step 608, the method 600 may include assessing user activity for a period of interest. User activity for the period of interest may be assessed based on the movement score calculated as described above. An assessment of user activity can include conclusions, coaching, and the like based on the movement score, and may be augmented based on other contextual data such as physiological data for the user, movement data, a movement score, a recovery score, a sleep score, data from a third party, an input received from the user, and so forth.

In one aspect, the assessment may include an evaluation of whether the movement score is above or below a mean for a population or a particular demographic within a population. The assessment may also or instead provide a comparison of movement score components to population norms, e.g., by indicating whether a user has inactive periods of greater frequency or duration, active periods of greater frequency, duration, or intensity, and so forth. In another aspect, the movement score and other movement data may be used to evaluate whether a user's physical activity is contributing to an increase or decrease in expected lifespan relative to an average for a population, or whether this data indicates a higher or lower all-cause mortality over some predetermined window (e.g., 10 years, 15 years, or some other range). The assessment may be used, for example, to provide user information and coaching as described herein.

In one aspect, assessing user activity for the period of interest may include generating one or more quantitative or categorical indicators derived from the movement score and associated metrics. For example, the movement score may be compared to one or more predetermined thresholds to classify user activity as low, moderate, or high for the period of interest. In another embodiment, the movement score may be normalized relative to a stored population distribution to determine a percentile ranking for the user within a selected demographic group. The demographic group may be defined according to age, sex, geographic region, fitness level, or other user attributes. Such comparisons may be performed using stored statistical models, including mean values, standard deviations, or other distribution parameters maintained in a local or remote database.

In one aspect, assessment of user activity may include evaluating trends over a period of time in the movement score and its component metrics. For example, the system may calculate a moving average of the movement score over a plurality of prior periods of interest and determine whether the user's activity level is increasing, decreasing, or remaining stable over time. Variability metrics, such as a standard deviation of movement score across multiple days, may also be calculated to assess consistency of activity patterns. These longitudinal assessments may provide additional context beyond a single-period score and may be used to detect sustained inactive behavior or progressive improvement.

In an embodiment, the movement score and related metrics may be mapped to one or more health risk models. For example, the movement score may serve as an input to a regression model, survival model, or other predictive model derived from epidemiological data to estimate a relative risk of adverse health outcomes over a predetermined time horizon. The output of such a model may include an estimated risk index, a relative risk ratio, or an estimated change in expected lifespan relative to a reference population. These outputs may be provided to the user in numerical or categorical form and may be used to generate personalized recommendations or coaching interventions. These outputs may also or instead be converted into an effective age that represents an all-cause mortality risk associated with activity patterns represented by the movement score.

In another aspect, assessment of the movement score may take into account a goal specified by the user. The goal may include, without limitation, a target movement score, a target maximum inactive period, a target amount of active time within a period of interest, or a target frequency or intensity of activity. The system may compare the calculated movement score and associated metrics to the user-defined goal to determine a degree of progress toward the goal. For example, the assessment may include determining whether the user is meeting, exceeding, or falling short of the specified target, and may further quantify a remaining amount of activity or reduction in inactive time required to achieve the goal within the current period of interest. In another embodiment, the goal may influence interpretation of the movement score by adjusting thresholds used to classify performance levels or by weighting particular components of the score in accordance with the stated objective. By incorporating user-defined goals into the assessment process, the system may provide feedback and coaching that is aligned with the user's intended outcomes while maintaining consistency with the underlying movement score framework. The goals and/or the resulting assessment of the user goals may also be used to modify specific targets, metrics, and thresholds, e.g., a target maximum inactive period, weighting of variables, movement score alert thresholds, and so forth.

By incorporating statistical normalization, trends over time, and predictive risk modeling into the assessment process, the system may generate an evaluation of user activity that is derived from structured computational analysis rather than raw movement metrics alone. Such processing may transform sensor data and derived movement scores into normalized indices, trend indicators, and risk-related outputs that are statistically grounded and suitable for comparative analysis across time and across populations. The resulting assessment may support and enable automated generation of feedback, alerts, and coaching recommendations based on quantitatively determined activity patterns and associated health risk correlations.

In some embodiments, the movement score and one or more associated component metrics (e.g., maximum inactive period, target inactive period, and total active time) may be transformed into a natural language assessment of user activity by applying rule-based logic, statistical normalization, generative AI (e.g., using large language models), and/or predictive modeling to generate one or more messages or message templates populated with user-specific values. For example, the system may compare the movement score to predetermined thresholds to assign a categorical performance band (e.g., low, moderate, high) and select corresponding narrative language describing whether activity patterns for the period of interest are supportive of the user's goals. The system may further identify primary drivers of the score by evaluating which component metric deviates most from its target (e.g., determining that the maximum inactive period exceeded the target inactive period by the largest margin) and generating explanatory text describing why the movement score is limited and what behavior would most efficiently improve it. In some embodiments, the assessment may include progress-to-goal language (e.g., remaining active minutes needed to reach a target score), comparisons to a user's historical baseline (e.g., relative to a rolling 7-day average), or comparisons to population norms (e.g., percentile rank within a demographic group), thereby converting computational outputs into a user-interpretable summary.

In some embodiments, the natural language assessment may be generated in real-time or near real-time as the movement score is progressively updated, and may include an actionable recommendation selected based on the detected activity pattern. For example, when the system determines that the movement score is being reduced primarily by a prolonged inactive bout, the system may generate an assessment such as: “Your movement score is trending low today because your longest inactive stretch reached 1 hour 50 minutes, which is above your 60-minute target; take a 5-10 minute walk to break up the next sitting period.” As another example, when the system determines that the base score is below target due to a long inactive period but the scaling factor indicates substantial compensatory activity, the system may generate an assessment such as: “You had one long stretch of sitting (about 90 minutes), but you've already accumulated 55 active minutes, which is helping you recover your score; keep adding short movement breaks to finish the day strong.” In this manner, the system can convert quantitative outputs into concise, up-to-date (e.g., real-time or near real-time), context-aware language describing current performance, principal contributors, and a suggested next action.

As shown in step 610, the method 600 may include alerting the user. An alert to the user may include a notification to the user from the fitness monitor such as a haptic notification, an audible notification (such as a beep), or a visible notification (such as a flashing LED). The alert may also or instead include a message, in-app notification, alert, or the like to a user device such as a smartphone. In another aspect, the alert may include an email or other message or notification that is accessible to the user through an application, website, or other resource.

Alerting a user may include generating or initiating an alert when the movement score meets a predetermined threshold. Alerting the user may include displaying results such as the data and scores to the user. Alerting the user may include alerting the user to provide coaching to the user. Displaying the data and scores to the user may include displaying the movement score to the user. Displaying the data and scores to the user may include displaying the maximum inactive period to the user. Displaying the data and scores to the user may include displaying the total active time over the period of interest to the user. Displaying the data and scores to the user may include displaying the total inactive time for the period of interest to the user. Displaying the data and scores to a user may include displaying at least one of the following: physiological data, movement data, a movement score, a recovery score, a sleep score, data from a third party, and an input received by the user. Other data may also or instead be displayed, such as any of the data from the assessment above, as well as raw data describing, e.g., mean activity time, mean inactive time, total inactive time, minutes of activity, and so forth. Descriptive or comparative data may also or instead be displayed, such as data comparing the user to a general population or a demographic group, data comparing the user's current performance to past performance for the movement score, a moving average of the movement score for the user, a graph of movement score over time, either for a day or for a multi-day interval such as a week or a month, and so forth.

As shown in step 612, the method 600 may include providing coaching to the user. Providing coaching to the user may include providing coaching to the user related to how to improve the movement score during a current period of interest. Providing coaching to the user may include providing coaching to the user related to how to improve the movement score for a subsequent period of interest. Providing coaching to a user may include at least one of the following: providing a recommendation to the user to more frequently break up inactive periods, providing recommended activities for a user to increase their movement score, providing a recommendation to the user to increase the intensity, duration, and/or frequency of activities, and providing recommendations to a user to improve recovery. The coaching may be provided based on the movement score, as well as other physiological or derived data such as heart rate data, strain data, respiration data, heart rate variability data, motion data, a recovery score, a sleep score, user input, a user goal, third party data, user assessment data, and so forth.

In one aspect, coaching may be provided on demand, or on some regular reporting schedule, e.g., daily, and may contain specific recommendations for improving the user's movement score in a subsequent time period. In another aspect, coaching may be delivered incrementally to a user. For example, a wearable monitor may flash or vibrate when a user is approaching the maximum recommended inactive period, or when coaching information is available. A corresponding message may be delivered to a phone, smart watch, display, or other user device to inform the user of the prolonged inactive state and/or to recommend an activity to break up the inactive state. More generally, any observations, recommendations, or guidance that might usefully improve short-term or long-term trends in the movement score may be used to provide coaching as described herein.

In one aspect, providing coaching to the user may include generating one or more personalized recommendations based on a comparison between the user's movement score and one or more thresholds, goals, or historical benchmarks. For example, the system may determine that the user's maximum inactive period exceeds the target maximum inactive period and may provide a recommendation to interrupt inactive time at a specified interval, such as standing, walking, or performing a brief activity. In another embodiment, the system may determine that the total active time for the current period of interest is below a target value and may recommend a specific duration or intensity of activity required to achieve a desired movement score within the current period of interest.

In one aspect, coaching may be dynamically adapted based on real-time or near real-time updates to the movement score. For example, as additional measurement intervals are classified and the movement score is progressively updated over the course of a day or other interval. The system may calculate a projected end-of-period movement score (e.g., for the day, or for a prolonged inactive period) based on current activity trends and/or comparisons to historical data. When the projected movement score is below a target, the system may provide proactive recommendations, such as suggesting a specific number of minutes of moderate activity within a remaining portion of the period of interest. Conversely, when the projected movement score exceeds a target in a good way, e.g., indicating a favorable activity profile, the system may provide reinforcement messaging or recommend recovery-oriented activities. In an embodiment, providing coaching may include selecting a recommended activity type based on historical user data. For example, the system may access stored data identifying activities previously performed by the user and associated movement score improvements. Based on this data, the system may recommend an activity that is correlated with efficient increases in overall movement score for that user. In another embodiment, recommended activities may be filtered based on contextual data, such as time of day, geographic location, weather data from a third party, calendar data, or equipment availability, thereby increasing the likelihood of user adherence.

In another aspect, coaching may include providing or modifying recommended intensity or duration of activity based on physiological data. For example, heart rate data, recovery scores, or sleep scores may be used to determine whether high-intensity activity is advisable. When recovery metrics indicate reduced readiness, the system may recommend low to moderate intensity activities that increase total active time without imposing excessive strain. When recovery metrics indicate high readiness, the system may recommend higher-intensity intervals that more rapidly increase the scaling factor applied to the base score. In this manner, coaching may balance activities associated with movement score improvement against overall physiological readiness.

Providing coaching may further include adjusting a frequency of notifications or interventions based on user engagement patterns. For example, if a user consistently responds to inactive alerts by initiating movement within a predetermined response window, the system may maintain or increase the frequency of reminders. Conversely, if a user frequently ignores alerts, the system may reduce alert frequency, adjust timing, or modify message content. Such adjustments may be based on stored engagement metrics, historical response times, or user preferences, thereby personalizing delivery of coaching.

In another aspect, coaching may be goal-oriented and may incorporate user-defined objectives. For example, a user may specify a target movement score, a maximum allowable inactive duration, or a weekly active time objective. The system may then calculate intermediate targets for the current period of interest and provide stepwise recommendations aligned with achieving the specified goal. For example, the system may determine that achieving the user's target movement score requires reducing the current maximum inactive period by a specified amount and may recommend structured breaks at defined intervals for the remainder of the day.

In one embodiment, providing coaching may include generating a summary report at the conclusion of a period of interest. The summary report may identify primary contributors to the movement score, such as a longest inactive interval or insufficient total active time, and may provide specific recommendations for a subsequent period of interest. For example, the report may indicate that the movement score was limited by a prolonged inactive interval during afternoon hours and may recommend scheduling predefined movement breaks during similar hours in a subsequent day.

In another aspect, coaching may incorporate predictive modeling to identify anticipated inactive periods based on historical behavior patterns. For example, if user data indicates that the user typically has extended inactive intervals during certain recurring calendar events or times of day, the system may preemptively provide guidance prior to the anticipated inactive interval. This may include recommending a brief activity immediately before the anticipated inactive period or scheduling automated reminders during the expected interval.

In one embodiment, providing coaching may include multi-modal delivery of recommendations through one or more devices. For example, coaching may be delivered via a haptic alert on a wearable monitor, a visual notification on a user device, an audible prompt, or a combination thereof. The modality may be selected based on user preference, environmental context, or urgency of the recommendation. For example, when the maximum inactive period is approaching a threshold associated with a significant reduction in the base score, a haptic alert may be generated to prompt immediate action.

In another aspect, coaching may include adaptive progression over multiple periods of interest. For example, when a user consistently achieves or exceeds a target movement score, the system may adjust one or more parameters, such as decreasing the target inactive period or increasing recommended active time, to promote gradual improvement. Conversely, when a user consistently fails to meet targets, the system may adjust targets to more attainable levels while maintaining incremental progression. Such adaptive progression may be based on historical averages, rolling windows of prior movement scores, or trend analyses derived from stored user data.

In general, providing coaching may include transforming raw sensor data and derived movement metrics into a movement score that supports actionable behavioral guidance tailored to the user. The recommendations may be generated using rule-based logic, statistical models, machine learning models, or combinations thereof, and may be updated with the movement score as new user data is received. By linking movement score components to personalized, context-aware recommendations, the system may facilitate short-term behavioral changes and long-term improvements in movement patterns over successive periods of interest.

As described herein, in some embodiments, coaching interactions informed by the movement score may be delivered through haptic feedback generated by a wearable monitor to prompt timely behavior changes that improve the movement score during a current period of interest. For example, the wearable monitor may continuously or periodically update the movement score based on identified inactive intervals and active intervals, determine when a current inactive bout is approaching a target inactive period or when the movement score is trending toward a predetermined low threshold, and initiate a haptic notification (e.g., a vibration pattern) to prompt the user to interrupt the inactive bout. The haptic notification may be graduated in urgency based on proximity to a threshold (e.g., a lighter vibration when the user is within 10 minutes of exceeding the target inactive period and a stronger vibration when the target is exceeded), and may be followed by additional haptic feedback confirming that a qualifying active interval has been detected and that the inactive bout has been broken. In this manner, the wearable monitor can provide an immediate, context-triggered coaching interaction that is driven by the movement score and designed to reduce prolonged inactive behavior and increase total active time. In this context, “immediate” coaching interactions will be understood to include coaching interactions that include timely interventions to correct a negative behavior while the negative behavior is occurring. In this manner, continuous physiological monitoring and calculation of resulting movement scores can facilitate real-time or near real-time delivery of contextual coaching feedback that improves the timing and effectiveness of user interventions.

In some embodiments, coaching informed by the movement score may leverage natural language techniques to improve user interactions by converting score components, trends, and projections into natural language explanations and actionable recommendations presented on a user device or within an application. A coaching engine may select message templates or generate text based on rules, statistical comparisons, and/or model-based inference regarding why the movement score is changing and what intervention is most likely to improve it, while tailoring tone and specificity based on user preferences, historical behavior, and time remaining in the period of interest. For example, when the movement score is limited primarily by a prolonged inactive interval, the system may present a message such as: “Your longest sitting stretch is 1 hour 30 minutes today, which is above your 60-minute target; a 6-minute walk now will reset the bout and protect your score.” When the movement score is suppressed by low total active time late in the day, the system may present a message such as: “You've logged 18 active minutes so far; adding two 10-minute brisk walks before 7 PM would meaningfully boost your score.” By providing explanations, prioritizing the most impactful next action, and adapting language to user context, natural language coaching can increase clarity, perceived relevance, and engagement with movement score-driven guidance.

It will be appreciated that these natural language interventions and suggestions may include multi-functional communications. That is, a message delivered via an in-app message, text message, or email, may include an assessment, e.g., a narrative description of the current user state, as described in step 608 above, an alert, e.g., a notification of a result or condition of interest, as described in step 610 above, and/or coaching feedback, e.g., advice or feedback concerning remediations, interventions, and future activities, as described in step 612 above. Alternatively, the assessment, alert, and coaching feedback may be provided separately, either through separate communication channels or in a timewise separated stream, or may be combined, sequenced, updated, and otherwise managed to improve movement scores or other user outcomes.

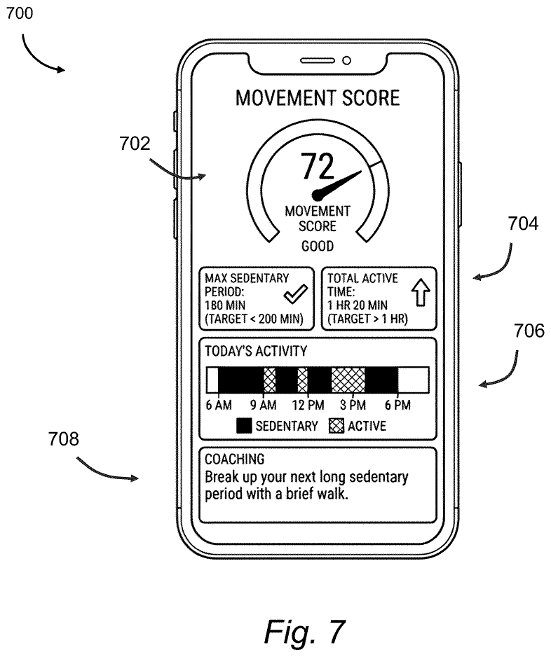

FIG. 7 shows a user interface 700 for presenting a movement score and associated metrics. A user interface 700 may be provided for presenting the movement score and associated metrics to a user during or after a period of interest. The user interface 700 may be displayed on a user device such as a mobile phone, tablet, smart watch, wearable monitor, or other computing device communicatively coupled to the wearable monitor described herein. The user interface 700 may include a graphical representation of the movement score for the period of interest, which may correspond to the movement score calculated as a function of the base score and scaling factor as described above.

In one aspect, the user interface 700 may display the movement score 702 as a numerical value, graphical gauge, progress indicator, or other visual element that conveys the user's current movement score relative to a target or reference value. The user interface 700 may further present one or more component metrics 704 used in calculating the movement score, including the maximum inactive period, a target maximum inactive period, total active time, weighted active time, and/or the base score and scaling factor. For example, the user interface may include a visual depiction of contiguous inactive intervals within the period of interest, along with an indication of whether the maximum inactive period exceeds the target maximum inactive period.

In another aspect, the user interface 700 may present a temporal distribution 706 of inactive and active measurement intervals over the period of interest. For example, a timeline or bar graph may display intervals classified as inactive and intervals classified as active based on motion data received from the wearable monitor. Such visualization may enable the user to identify prolonged inactive sequences and active interruptions thereof. The user interface 700 may also display the total active time accumulated thus far in the period of interest and may provide an indication of how additional active intervals may influence the scaling factor and overall movement score.

In another aspect, the user interface 700 may provide contextual feedback based on an assessment of the movement score. For example, the user interface 700 may indicate whether the movement score is above or below a population mean, percentile, or user-defined goal. The interface may further present projections of the movement score based on current trends within the period of interest and may indicate a remaining amount of active time required to achieve a target movement score. In an embodiment, the user interface 700 may provide interactive elements allowing a user to input contextual information, such as identification of an activity not automatically detected, which may trigger recalculation of interval classifications and updating of the movement score.

In another aspect, the user interface 700 may provide coaching 708, including prompts and recommendations derived from the movement score and associated metrics. For example, when a current inactive duration approaches or exceeds, the interface may generate a visual, auditory, or haptic notification recommending interruption of the inactive period. Similarly, when total active time is below a target threshold, the interface may recommend specific activities, durations, or intensity levels calculated to improve the scaling factor and movement score. In this manner, the user interface 700 may present structured movement score data, component metrics, and responsive feedback to support user awareness, engagement, and improvement in movement behavior.

In general, each of these components of the user interface 700 may include interactive controls or the like to support user investigation of the data supporting the movement score, data analysis, coaching recommendations, and so forth, so that a user can investigate the underlying support for information presented in the user interface. For example, in some embodiments, a movement score user interface may present the movement score for a period of interest along with supporting component metrics and contextual data that explain how the movement score was determined. For example, the user interface may display a current movement score and a target movement score, a base score derived from a maximum inactive period, a scaling factor derived from total active time, the maximum inactive period and a corresponding target inactive period, total active time (and, in some embodiments, weighted active time based on intensity), total inactive time, number of inactive bouts, number of activity breaks, and time remaining in the period of interest. The user interface may further present a timeline view (or heatmap) showing classifications of measurement intervals (inactive versus active) across the day, including identification of the longest inactive bout and one or more active interruptions that reset or truncate inactive bout duration, and may optionally include supporting physiology, such as heart rate trend overlays during intervals, detected intensity values, and data quality indicators (e.g., off-wrist intervals, motion artifact flags, or classification confidence values) that help the user interpret why particular intervals were classified as inactive or active.

In some embodiments, the user interface may provide comparisons and reasoning outputs derived from population statistics and historical user data. For example, the interface may display a percentile rank of the movement score within a selected demographic cohort, a comparison of the user's maximum inactive period and total active time to cohort medians, and a trend view comparing the current day to a rolling average (e.g., 7-day average) and to prior periods of interest. The interface may also present projections of an end-of-day movement score based on the user's current trajectory, and may identify primary drivers of the score by indicating whether the movement score is being limited primarily by an elevated maximum inactive period, insufficient total active time, or insufficient frequency of interruptions, and may further show “what-if” deltas indicating how a qualifying break (or additional active minutes) would be expected to change the movement score given the current state.

In some embodiments, the user interface may include interactive features that permit investigation of supporting data, reasoning, and comparisons. For example, the user may select (e.g., tap) a segment of the timeline to view the underlying interval classification details, including activity probabilities or confidence, supporting heart rate and motion traces, and the rule or threshold used to determine whether the interval qualified as active; the user may also add context (e.g., label an activity, indicate a nap, or correct an interval) and trigger recalculation of affected inactive bouts and the movement score. The user interface may further allow the user to switch comparison cohorts (e.g., age band, sex, fitness level, region, or a custom peer group), toggle overlays for population medians and percentile bands, and view a “why this score” explanation panel that traces the movement score to the maximum inactive period, total active time, base score, and scaling factor, including a breakdown of which specific bouts or blocks of time contributed most to score reduction or improvement. In some embodiments, the user interface may include a parameter exploration control that permits temporary adjustment of personalization targets (e.g., a target inactive period or an activity qualification threshold) to preview how different coaching targets would affect the score and to support weekly planning and goal setting.

In other embodiments, the systems and methods described herein may be used more generally to improve reporting of data-driven health states and recommended behavioral interventions as described, by way of non-limiting examples, in U.S. application Ser. No. 19/461,616 filed on Jan. 27, 2026, where measurable user metrics and behaviors are more generally associated with all-cause mortality data in order to inform calculations of an effective age indicative of a user's all-cause mortality risk, and to provide data-driven, user-specific interventions based on user behaviors and user metrics. Thus, for example, a movement score as described herein may usefully be integrated into the methods and systems described in U.S. application Ser. No. 19/461,616, and similarly, the analysis and coaching techniques described in U.S. application Ser. No. 19/461,616 may be used with the movement score described herein.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application-specific integrated circuits, programmable gate arrays, programmable array logic components, or any other device or devices that may be configured to process electronic signals. It will further be appreciated that a realization of the processes or devices described above may include computer-executable code created using a structured programming language such as C, an object-oriented programming language such as C++, or any other high-level or low-level programming language (including assembly languages, hardware description languages, and database programming languages and technologies) that may be stored, compiled, or interpreted to run on one of the above devices, as well as heterogeneous combinations of processors, processor architectures, or combinations of different hardware and software.

Thus, in one aspect, each method described above, and combinations thereof, may be embodied in computer-executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory, or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party, such as a remote user, a remote processing resource (e.g., a server or cloud computer), or a machine, to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

What is claimed is:

1. A computer program product comprising computer-executable code that, when executing on one or more computing devices, performs the steps of:
continuously receiving motion data from one or more motion sensors of a fitness monitor worn by a user over a period of interest;
performing a plurality of activity classifications with a machine learning model during the period of interest;
identifying inactive intervals and active intervals within the period of interest based on the plurality of activity classifications;
determining a maximum inactive period based on a greatest duration of contiguous inactive intervals during the period of interest;
determining a total active time based on a sum of durations of the active intervals during the period of interest;
calculating a movement score for the user based on a combination of the maximum inactive period for the user over the period of interest and the total active time for the user over the period of interest;
assessing user activity for the period of interest based on the movement score; and
generating an alert when the movement score meets a predetermined threshold.

2. The computer program product of claim 1, wherein the period of interest is a current day.

3. The computer program product of claim 1, wherein the alert includes a haptic notification to the fitness monitor.

4. The computer program product of claim 1, further comprising adjusting the movement score based on one or more of the plurality of activity classifications and one or more corresponding activity intensities detected based on data from the fitness monitor.

5. A method comprising:
receiving user data from one or more sensors of a monitor worn by a user over a period of interest;
identifying inactive intervals within the period of interest;
identifying active intervals within the period of interest;
determining a maximum inactive period based on a greatest duration of contiguous inactive intervals during the period of interest;
determining a total active time based on a sum of durations of the active intervals during the period of interest;
calculating a movement score for the user based on a combination of the maximum inactive period for the user over the period of interest and the total active time for the user over the period of interest; and
initiating a notification to the user based on the movement score.

6. The method of claim 5, further comprising displaying the movement score to the user.

7. The method of claim 5, further comprising displaying movement metrics to the user, including one or more of the maximum inactive period, the total active time, and the movement score.

8. The method of claim 5, further comprising providing coaching to the user on how to improve the movement score during a current day or a subsequent day.

9. The method of claim 5, wherein identifying the active intervals includes identifying motion patterns associated with known physical activity within the user data using a machine learning model.

10. The method of claim 9, wherein the machine learning model includes at least one of a gradient-boosted model for activity classification and a recurrent neural network with gated recurrent units for activity classification.

11. The method of claim 5, wherein the user data includes at least one of motion data and heart rate data.

12. The method of claim 5, further comprising reducing a volatility of a calculation of the movement score at a beginning of a day for the user by adjusting a weighting of a contribution of the total active time to the movement score at the beginning of the day based on a number of measurement intervals for the day.

13. The method of claim 5, wherein identifying the active intervals within the period of interest includes:
performing an activity classification with a machine learning model a number of times during a measurement interval within the period of interest to provide a number of activity detections for the measurement interval; and
identifying the measurement interval as an active interval when the number of activity detections for the measurement interval meets a predetermined threshold.

14. The method of claim 5, wherein calculating the movement score includes calculating a base score for the period of interest using a function based on a difference between the maximum inactive period and a target inactive period.

15. The method of claim 5, wherein calculating the movement score includes calculating a base score for the period of interest using a first function that decreases as the maximum inactive period for the user increases.

16. The method of claim 15, wherein calculating the movement score includes calculating a scaling factor for the period of interest using a second function that increases as the total active time increases.

17. The method of claim 16, wherein calculating the movement score includes multiplying the base score by the scaling factor.

18. The method of claim 5, wherein identifying the active intervals within the period of interest includes:
performing an activity classification with a classification model a number of times over a measurement interval;
dividing the measurement interval into a number of sub-intervals;
identifying each of the number of sub-intervals as an active sub-interval when a number of activity detections in each of the number of sub-intervals meets a first predetermined threshold to provide a number of active sub-intervals; and
identifying the measurement interval as an active interval when the number of active sub-intervals in the measurement interval meets a second predetermined threshold.

19. The method of claim 5, further comprising progressively updating the movement score over a day for the user.

20. The method of claim 5, wherein the one or more sensors include at least one accelerometer.

21. The method of claim 5, wherein the one or more sensors include at least one gyroscope.

22. The method of claim 5, wherein the one or more sensors include at least one optical sensor.

23. The method of claim 5, wherein the one or more sensors include at least one electrical sensor.

24. The method of claim 5, wherein the one or more sensors include at least one audio sensor.

25. A system comprising:
a physiological monitor including one or more sensors configured to continuously acquire user data for a user with the one or more sensors; and
one or more processors configured by computer-executable code to receive data from the physiological monitor and to perform the steps of:
receiving motion data from the one or more sensors over a period of interest,
receiving heart rate data from the one or more sensors over the period of interest,
identifying inactive intervals and active intervals within the period of interest based on a combination of the motion data and the heart rate data,
determining a maximum inactive period based on a greatest duration of contiguous inactive intervals during the period of interest,
determining a total active time based on a sum of durations of the active intervals during the period of interest,
calculating a movement score for the user based on a combination of the maximum inactive period for the user over the period of interest and the total active time for the user over the period of interest, and
initiating an alert to the user when the movement score meets a predetermined threshold.

26. The system of claim 25, wherein the one or more processors include a processor in the physiological monitor.

27. The system of claim 25, wherein the one or more processors include a processor of a remote server coupled in a communicating relationship with the physiological monitor through a data network.

28. The system of claim 25, wherein the physiological monitor includes a wrist-worn monitor or a ring.
